(19) United States
(12) Patent Application Publication
(10) Pub. No.: US 2008/0135047 A1
(43) Pub. Date: Jun. 12, 2008

(54) Methods and Devices For Assisting Drug Delivery to the Lungs

(76) Inventors: Douglas Johnson, Boston, MA (US); Ronald Dalbec, Marlborough, MA (US)

Correspondence Address: NUTTER MCCLENNEN & FISH LLP, WORLD TRADE CENTER WEST, 155 SEAPORT BOULEVARD, BOSTON, MA, 02210-2604, US

(21) Appl. No.: 11/569,185

(22) PCT Filed: Jun. 3, 2005
(86) PCT No.: PCT/US05/19502

§ 371 Date: Sep. 13, 2007

Related U.S. Application Data
(60) Provisional application No. 60/521,672, filed on Jun. 16, 2004.

(51) Int. Cl. A61M 15/00

(52) U.S. Cl. 128/203.15

(57) ABSTRACT

Methods and devices for assisting in the delivery of medicaments to the lungs of a patient are provided using a non-aerosol, inhalation device. In one embodiment, the methods and device utilize a positive pressure device that is effective to drive a medicament contained within a chamber in an inhalation device through an outlet and into a patient's lungs, either orally, through the patient's nasal passages, or through a tracheal tube in communication with the patient's lungs. In another embodiment, the present invention provides a connector for coupling an inhalation device to a tracheal tube to allow a medicament contained within a drug chamber in the inhalation device to be delivered to the patient's lungs via the tracheal tube.

### BACKGROUND OF THE INVENTION

Many patients rely on inhalant-based medications to treat emphysema, asthma, or other lung deficiencies or airway impairments. Many different types of inhalers can be used to delivery the medications, including dry powder inhalers, suspension metered dose inhalers, suspension nebulisers, and suspension atomizers. The latter three types of inhalers utilize an aerosol to pump the medication from the inhaler into the patient's lungs. Since these inhalers do not require the patient to inhale in order to receive the medication, these inhalers can be used for delivering drugs through a tube extending into a patient's trachea.

Recently, dry powder inhalers have become more popular due to increasing environmental concerns over the use of aerosols contained in suspension metered dose inhalers, suspension nebulisers, and suspension atomizers. Dry powder inhalers typically contain a chamber for receiving a receptacle containing a drug, an implement for puncturing the receptacle to release the drug, and a mouth piece or nose piece for inhalation of the drug through the mouth or nose. In use, the patient is required to inhale when the implement is activated to draw the drug from the chamber into their lungs. Accordingly, current dry powder inhalers are limited to oral or nasal use.

Accordingly, there remains a need for methods and devices for delivering dry powder medicaments to patients that have difficulty inhaling, and to patient's that have a tracheal tube.

### SUMMARY OF THE INVENTION

The present invention provides methods and device for facilitating the delivery of a dry powder medicament to a patient's lungs, either orally or through a tracheal tube. In one embodiment, an inhalation device is provided having a drug chamber with an inlet and an outlet, and an actuation mechanism that is effective to release a medicament into the chamber. The device also includes a positive pressure generating device that is coupled to the inlet in the drug chamber and that is effective to selectively pump the medicament from the drug chamber through the outlet after the medicament is released. The positive pressure device can be directly coupled to the inhalation device, or a hollow elongate tube can extend from the positive pressure device to the inlet in the chamber of the inhalation device. In use, the positive pressure generator eliminates the need for the patient to inhale in order to receive the medicament.

In one embodiment, the inhalation device can include a mouthpiece that is in communication with the outlet in the chamber. The mouthpiece can be adapted to be positioned between a patient's lips to allow oral delivery, or it can be configured for nasal delivery. In another embodiment, the inhalation device can include a mouthpiece connector that has a first end that is adapted to couple to the mouthpiece and a second end that is adapted to couple to a tracheal tube in communication with a patient's lungs. In use, the mouthpiece connector allows the patient to inhale through the tracheal tube to receive the medicament, or optionally the positive pressure device can be used in combination with the mouthpiece connector to deliver the medicament to the patient's lungs via the tracheal tube.

The present invention also provides a method for delivering a dry powder medicament to the lungs. In general, the method includes the steps of activating an actuation mechanism on an inhalation device to cause the actuation mechanism to release a medicament into a chamber in the inhalation device, and activating a positive pressure generating device in communication with an inlet in the chamber of the inhalation device to cause the medicament to flow from the chamber through an outlet in the chamber and into a patient's lungs.

In other aspects of the present invention, a method for delivering a dry powder medicament to the lungs is provided and it generally includes the steps of coupling an outlet in an inhalation device to a tracheal tube, in communication with a person's lungs, and activating an actuation mechanism on the inhalation device to cause the actuation mechanism to release a medicament into a chamber in the inhalation device such that, when the patient inhales, the medicament flows from the chamber through the outlet and into the tracheal tube to be delivered to the patient's lungs. A positive pressure generating device can optionally be used to deliver the medicament to the tracheal tube. In another embodiment, the inhalation device can include a mouthpiece that is in communication with the outlet in the drug chamber. A mouthpiece connector can be provided for allowing the mouthpiece to couple to a tracheal tube.

In yet another embodiment of the present invention, a kit is provided for use with a inhalation device having a drug chamber adapted to receive a medicament, and an actuation mechanism effective to release the medicament into the chamber. The kit can include a positive pressure generating device having a hollow elongate tube coupled thereto and including a terminal end that is adapted to mate to an inlet in communication with the drug chamber in the inhalation device. An inhalation device can also optionally be provided. The kit can also optionally or alternatively include a mouthpiece connector having a first end that is adapted to couple to a mouthpiece formed on the inhalation device, and a second end that is adapted to couple to a tracheal tube in communication with a patient's lungs.

### DETAILED DESCRIPTION OF THE DRAWINGS

The invention will be more fully understood from the following detailed description taken in conjunction with the accompanying drawings, in which:

FIG. 1 is a side, partially transparent view of one embodiment of a prior art inhalation device for use with the present invention;

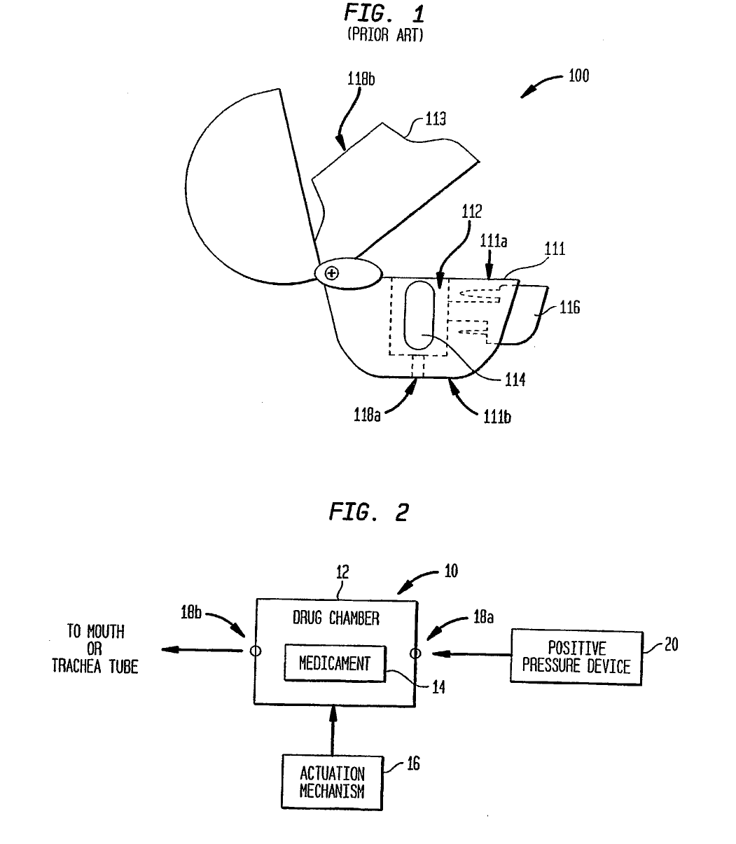

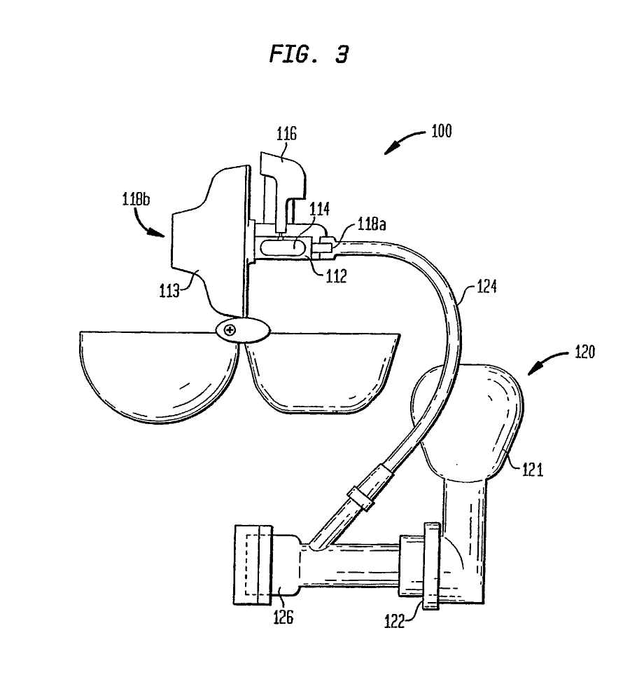

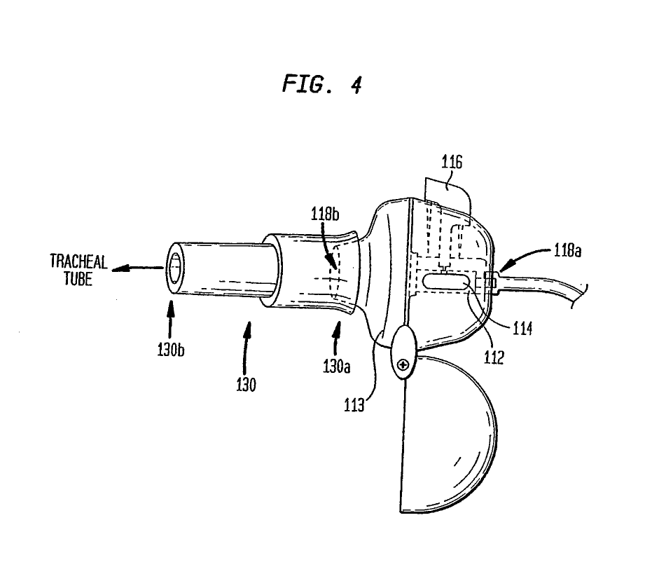

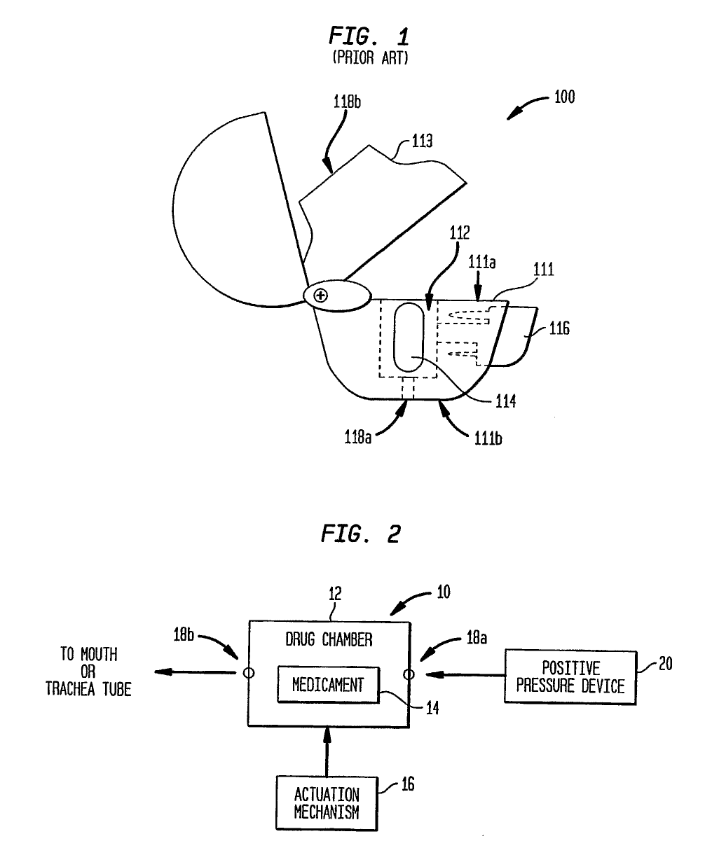

FIG. 2 is a schematic illustration of one embodiment of a system for delivering a medicament to a patient's lungs;

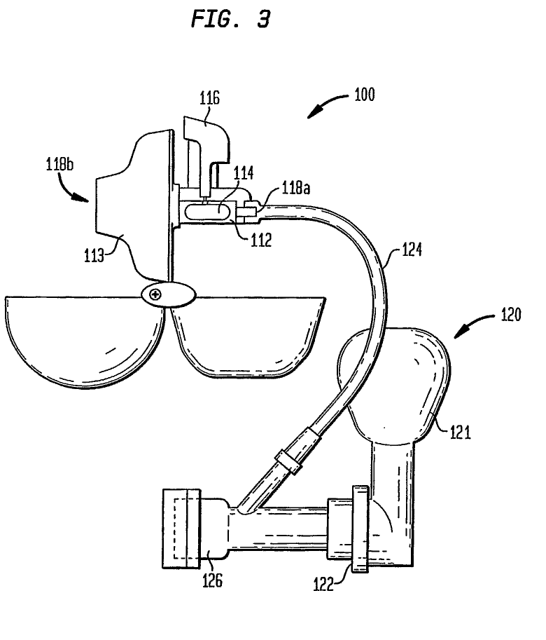

FIG. 3 is a side, partially transparent view illustration of the prior art inhalation device shown in FIG. 1 coupled to a positive pressure device; and

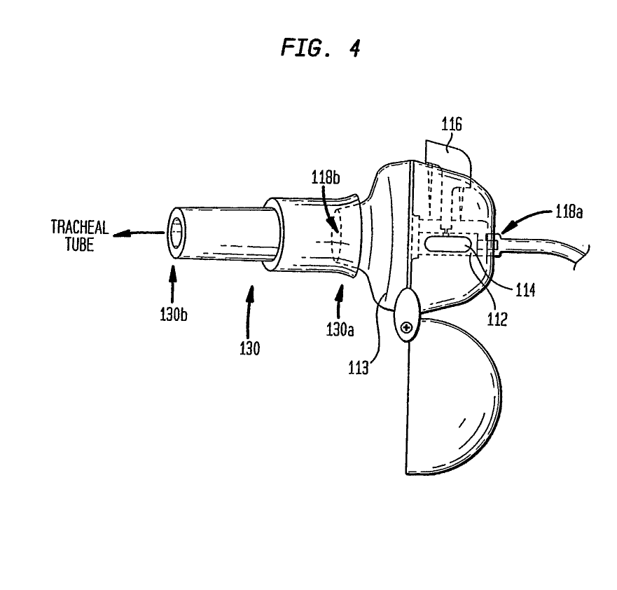

FIG. 4 is a side, partially transparent view illustration of the prior art inhalation device shown in FIG. 1 coupled to connector for mating the device to a tracheal tube.

### DETAILED DESCRIPTION OF THE INVENTION

FIG. 1 illustrates a prior art inhalation device 100 that is similar to the Handihaler™. The device 100 includes a drug chamber 112 that is formed within a housing 111 and that is adapted to receive a medicament 114 that is in the form of a capsule. An actuation mechanism 116 is operably coupled to the housing 111 and the drug chamber 112 such that, when the actuation mechanism 116 is depressed, it is effective to puncture the capsule, thereby releasing the medicament 114 into the drug chamber 112. A person skilled in the art will appreciate that the terms “release” and “releasing,” as used herein, are intended to include devices in which the actuation mechanism 116 merely punctures the capsule without causing the medicament to flow from the capsule, as well as those devices wherein the medicament flows out of the capsule or otherwise flows into the drug chamber 112. The inhalation device 100 also includes an inlet 118a and an outlet 118b, each of which are configured to communicate with the drug chamber 112. In the illustrated embodiment, the outlet 118b is formed in a mouthpiece 113 that mates to one end 111a of the housing 111, and the inlet 118a is formed in an opposite end 111b of the housing 111. The mouthpiece 113 is shown in an open configuration separated from the housing 111 to allow the encapsulated medicament 114 to be disposed within the drug chamber 112. When the mouthpiece 113 is closed, a passageway (not shown) within the mouthpiece communicates with the drug chamber 112 to allow the medicainent 114 to flow therethrough. The inhalation device 100 can also include a variety of other features that are not shown or illustrated in the drawings. By way of non-limiting example, a porous member, such as a grate, can be placed between the drug chamber 112 and the mouthpiece 113 to prevent the capsule from passing into the mouthpiece 113.

When the device is used as normally intended, the medicament 114 is disposed within the drug chamber 112 and the mouthpiece 113 is closed. The actuation mechanism 116 is then depressed to puncture the capsule, thereby releasing the medicament 114 into the chamber 114. The patient then places their mouth over the mouthpiece 113 and inhales to inhale the medicament 114 from the drug chamber 112 through the outlet 118b and into their lungs. The air inlet 118a allows air to flow therethrough as the patient inhales. While the device 100 can be effective for certain patients, it cannot be used by patients who cannot take deep breaths, or by patients having a tracheal tube implanted.

Accordingly, the present invention provides techniques for modifying the inhalation device to allow a medicament to be delivered to a patient orally or through the nasal passages without requiring the patient to inhale, or to allow the medicament to be delivered to a tracheal tube. FIG. 2 is a schematic illustration of one possible configuration of a device or system in accordance with the present invention. As shown, 4 the system generally includes an inhalation device 10 having a drug chamber 12 that is adapted to receive a medicament 14, and an actuation mechanism 16 that is adapted to release the medicament 14 into the drug chamber 12. In one embodiment, an inlet 8a in the drug chamber 12 is coupled with a positive pressure device 20 for creating a positive pressure within the drug chamber 12. The drug chamber 12 also includes an outlet 18b for allowing the medicament to be delivered to a patient's lungs after the actuation mechanism 16 and the positive pressure device 20 are both activated. In another embodiment, the device 10 can include a connector (not shown) for coupling the outlet 18b in the drug chamber 12 to a tracheal tube. Where the inhalation device 10 includes a mouthpiece in communication with the outlet 18b, the connector can mate directly to the mouthpiece. The connector can be used alone with the inhalation device 10, or optionally it can be used in combination with the positive pressure device 20.

FIGS. 3-4 illustrate the inhalation device 100 of FIG. 1 as modified in accordance with the system shown in FIG. 2. Referring to FIG. 3 first, the air inlet 118a on the inhalation device 100 can be coupled to a positive pressure device 120 that is effective to create a positive pressure within the drug chamber 112 to force the medicament 114 through the outlet 118b in the drug chamber 112. A person skilled in the art will appreciate that virtually any positive pressure device can be used, including both manual and automatic devices for creating a positive pressure. By way of non-limiting example, FIG. 3 illustrates one exemplary embodiment of a positive pressure device 120 that is in the form of a standard ambulatory respiration device. The device 120 generally includes a balloon or bag member 121 having an outlet port 122 formed therein. Depending on the particular configuration of the inlet 118a formed in the inhalation device 100, the outlet 122 can either be directly connected to the inlet 118a, or it can be connected via a hollow elongate tube 124, as shown. The hollow elongate tube 124 should have a diameter that is sufficient to allow the terminals ends of the tube 124 to form a seal with the outlet 122 on the positive pressure device 120 and with the air inlet 118a on the inhalation device 100. Various connectors, tubing, or other mechanisms can also or alternatively be used to connect the positive pressure device 120 to the outlet 122 on the positive pressure device and/or to the inlet 118a in the inhalation device 100. For example, as is further shown in FIG. 3, an adapter 126 is provided and it extends between the outlet 122 on the positive pressure device 120 and the terminal end of the hollow elongate tube 124. Regardless of the type of connection formed between the positive pressure device 120 and the inhalation device 100, the connection should preferably be sealed to prevent leakage and to allow sufficient air pressure to be provided to the drug chamber 112 in the inhalation device 100.

The outlet 118b of the inhalation device 100 can be configured to deliver the medicament orally or to the patient's nasal passage, or it can be configured to connect to a tracheal tube. In the embodiment shown in FIG. 3, the inhalation device 100 includes a mouthpiece 113 that allows the patients to place their lips there around to form a seal. Thus, when the actuation mechanism 116 is depressed, and the positive pressure device 120 is activated, the medicament 114 is forced through the outlet 118b and the mouthpiece 113 to be delivered through the patient's mouth to their lungs. A person skilled in the art will appreciate that the mouthpiece 113 can have virtually any configuration, and that the mouthpiece 113 can alternatively be configured to deliver the medicament to a patient's nasal passage.

In use, a medicament 114 is placed within the drug chamber 112, and the actuation mechanism 116 is depressed to release the medicament from the capsule. The balloon or bag 121 is compacted or squeezed to blow air through the outlet 122 and the hollow elongate tube 124 and into the inlet 118a of the inhalation device 100, thereby creating a positive pressure within the drug chamber 112 to force the medicament 114 out through the outlet 118b and into the patient's lungs. In the embodiment shown in FIG. 3, the medicament 114 flows through the mouthpiece 113 and into the patient's mouth.

In an another embodiment, shown in FIG. 4, the inhalation device 100 can be adapted to connect to a tracheal tube. By way of non-limiting example, FIG. 4 illustrates a generally hollow connector 130 having first and second ends 130a, 130b that extend between the outlet 118b of the inhalation device 100 and a tracheal tube (not shown). The first end 130a can have a variety of configurations depending on the configuration of the outlet 118b in the inhalation device 100. In the illustrated embodiment, however, the first end 130a is adapted to mate to the mouthpiece 113, and more particularly it fits around the mouthpiece 113 to form a seal therewith. The second end 130b is similarly configured to mate to a tracheal tube, and it preferably simply slides over the tube to form a seal therewith.

In use, the connector 130 is mated to the mouthpiece 113 and a tracheal tube. The actuation mechanism 116 is then depressed, and the patient either inhales to cause the medicament 114 to flow through the mouthpiece 113 and the connector 130 and into a tracheal tube. In an alternative embodiment, where the patient cannot inhale, the connector 130 can be used in combination with the positive pressure device 120.

A person skilled in the art will appreciate that, while FIGS. 3 and 4 illustrate an inhalation device that is similar to the Handihaler™, a variety of inhalation devices can be used with the present invention. Moreover, the configuration of the inhalation device 10, and in particular the drug chamber 12 and the actuation mechanism 16 of the inhalation device 10, can vary depending on the configuration of the medicament 14. For example, in one embodiment the medicament 14 can be contained within a capsule or other receptacle that is placed within the drug chamber 12. The actuation mechanism 16 can thus be effective to puncture the capsule or otherwise cause the capsule to release the medicament 14 therefrom. In another embodiment, the medicament does not need to be encapsulated, but rather it can be contained outside of the drug chamber 12. The actuation mechanism can thus be configured to release a predetermined dosage of the medicament 14 into the drug chamber 12. A person skilled in the art will appreciate that the inhalation device 10 can have a variety of configurations, and that virtually any inhalation device can be used with the present invention. Certain modifications may be necessary, however, depending on the particular inhalation device used.

In an exemplary embodiment, the inhalation device 10 is preferably adapted to deliver a dry powder medicament. By way of non-limiting example, suitable dry powder inhalers for use with the methods and devices of the present invention include, but are not limited to, capsule devices such as Spinhaler™, Rotahaler™, Handihaler™, Aerohaler™, Eclipse™, Turbospin™ and the Flowcaps™ inhaler, and inultidose inhalers such as Accuhaler™, Turbuhaler™, Ultrahaler™, Diskhaler™, Novoliser™, Easyhaler™, Taifun™, Clickhaler™, Twisthaler™ and Aspirair™.

One of ordinary skill in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

### BACKGROUND OF THE INVENTION

Many patients rely on inhalant-based medications to treat emphysema, asthma, or other lung deficiencies or airway impairments. Many different types of inhalers can be used to delivery the medications, including dry powder inhalers, suspension metered dose inhalers, suspension nebulisers, and suspension atomizers. The latter three types of inhalers utilize an aerosol to pump the medication from the inhaler into the patient's lungs. Since these inhalers do not require the patient to inhale in order to receive the medication, these inhalers can be used for delivering drugs through a tube extending into a patient's trachea.

Recently, dry powder inhalers have become more popular due to increasing environmental concerns over the use of aerosols contained in suspension metered dose inhalers, suspension nebulisers, and suspension atomizers. Dry powder inhalers typically contain a chamber for receiving a receptacle containing a drug, an implement for puncturing the receptacle to release the drug, and a mouth piece or nose piece for inhalation of the drug through the mouth or nose. In use, the patient is required to inhale when the implement is activated to draw the drug from the chamber into their lungs. Accordingly, current dry powder inhalers are limited to oral or nasal use.

Accordingly, there remains a need for methods and devices for delivering dry powder medicaments to patients that have difficulty inhaling, and to patient's that have a tracheal tube.

### SUMMARY OF THE INVENTION

The present invention provides methods and device for facilitating the delivery of a dry powder medicament to a patient's lungs, either orally or through a tracheal tube. In one embodiment, an inhalation device is provided having a drug chamber with an inlet and an outlet, and an actuation mechanism that is effective to release a medicament into the chamber. The device also includes a positive pressure generating device that is coupled to the inlet in the drug chamber and that is effective to selectively pump the medicament from the drug chamber through the outlet after the medicament is released. The positive pressure device can be directly coupled to the inhalation device, or a hollow elongate tube can extend from the positive pressure device to the inlet in the chamber of the inhalation device. In use, the positive pressure generator eliminates the need for the patient to inhale in order to receive the medicament.

In one embodiment, the inhalation device can include a mouthpiece that is in communication with the outlet in the chamber. The mouthpiece can be adapted to be positioned between a patient's lips to allow oral delivery, or it can be configured for nasal delivery. In another embodiment, the inhalation device can include a mouthpiece connector that has a first end that is adapted to couple to the mouthpiece and a second end that is adapted to couple to a tracheal tube in communication with a patient's lungs. In use, the mouthpiece connector allows the patient to inhale through the tracheal tube to receive the medicament, or optionally the positive pressure device can be used in combination with the mouthpiece connector to deliver the medicament to the patient's lungs via the tracheal tube.

The present invention also provides a method for delivering a dry powder medicament to the lungs. In general, the method includes the steps of activating an actuation mechanism on an inhalation device to cause the actuation mechanism to release a medicament into a chamber in the inhalation device, and activating a positive pressure generating device in communication with an inlet in the chamber of the inhalation device to cause the medicament to flow from the chamber through an outlet in the chamber and into a patient's lungs.

In other aspects of the present invention, a method for delivering a dry powder medicament to the lungs is provided and it generally includes the steps of coupling an outlet in an inhalation device to a tracheal tube in communication with a person's lungs, and activating an actuation mechanism on the inhalation device to cause the actuation mechanism to release a medicament into a chamber in the inhalation device such that, when the patient inhales, the medicament flows from the chamber through the outlet and into the tracheal tube to be delivered to the patient's lungs. A positive pressure generating device can optionally be used to deliver the medicament to the tracheal tube. In another embodiment, the inhalation device can include a mouthpiece that is in communication with the outlet in the drug chamber. A mouthpiece connector can be provided for allowing the mouthpiece to couple to a tracheal tube.

In yet another embodiment of the present invention, a kit is provided for use with a inhalation device having a drug chamber adapted to receive a medicament, and an actuation mechanism effective to release the medicament into the chamber. The kit can include a positive pressure generating device having a hollow elongate tube coupled thereto and including a terminal end that is adapted to mate to an inlet in communication with the drug chamber in the inhalation device. An inhalation device can also optionally be provided. The kit can also optionally or alternatively include a mouthpiece connector having a first end that is adapted to couple to a mouthpiece formed on the inhalation device, and a second end that is adapted to couple to a tracheal tube in communication with a patient's lungs.

### DETAILED DESCRIPTION OF THE DRAWINGS

The invention will be more fully understood from the following detailed description taken in conjunction with the accompanying drawings, in which:

FIG. 1 is a side, partially transparent view of one embodiment of a prior art inhalation device for use with the present invention;

FIG. 2 is a schematic illustration of one embodiment of a system for delivering a medicament to a patient's lungs;

FIG. 3 is a side, partially transparent view illustration of the prior art inhalation device shown in FIG. 1 coupled to a positive pressure device; and

FIG. 4 is a side, partially transparent view illustration of the prior art inhalation device shown in FIG. 1 coupled to connector for mating the device to a tracheal tube.

### DETAILED DESCRIPTION OF THE INVENTION

FIG. 1 illustrates a prior art inhalation device 100 that is similar to the Handihaler™. The device 100 includes a drug chamber 112 that is formed within a housing 111 and that is adapted to receive a medicament 114 that is in the form of a capsule. An actuation mechanism 116 is operably coupled to the housing 111 and the drug chamber 112 such that, when the actuation mechanism 116 is depressed, it is effective to puncture the capsule, thereby releasing the medicament 114 into the drug chamber 112. A person skilled in the art will appreciate that the terms “release” and “releasing,” as used herein, are intended to include devices in which the actuation mechanism 116 merely punctures the capsule without causing the medicament to flow from the capsule, as well as those devices wherein the medicament flows out of the capsule or otherwise flows into the drug chamber 112. The inhalation device 100 also includes an inlet 118a and an outlet 118b, each of which are configured to communicate with the drug chamber 112. In the illustrated embodiment, the outlet 118b is formed in a mouthpiece 113 that mates to one end 11 la of the housing 111, and the inlet 118a is formed in an opposite end 111b of the housing 111. The mouthpiece 113 is shown in an open configuration separated from the housing 111 to allow the encapsulated medicament 114 to be disposed within the drug chamber 112. When the mouthpiece 113 is closed, a passageway (not shown) within the mouthpiece communicates with the drug chamber 112 to allow the medicament 114 to flow therethrough. The inhalation device 100 can also include a variety of other features that are not shown or illustrated in the drawings. By way of non-limiting example, a porous member, such as a grate, can be placed between the drug chamber 112 and the mouthpiece 113 to prevent the capsule from passing into the mouthpiece 113.

When the device is used as normally intended, the medicament 114 is disposed within the drug chamber 112 and the mouthpiece 113 is closed. The actuation mechanism 116 is then depressed to puncture the capsule, thereby releasing the medicament 114 into the chamber 114. The patient then places their mouth over the mouthpiece 113 and inhales to inhale the medicament 114 from the drug chamber 112 through the outlet 118b and into their lungs. The air inlet 118a allows air to flow therethrough as the patient inhales. While the device 100 can be effective for certain patients, it cannot be used by patients who cannot take deep breaths, or by patients having a tracheal tube implanted.

Accordingly, the present invention provides techniques for modifying the inhalation device to allow a medicament to be delivered to a patient orally or through the nasal passages without requiring the patient to inhale, or to allow the medicament to be delivered to a tracheal tube. FIG. 2 is a schematic illustration of one possible configuration of a device or system in accordance with the present invention. As shown, the system generally includes an inhalation device 10 having a drug chamber 12 that is adapted to receive a medicament 14, and an actuation mechanism 16 that is adapted to release the medicament 14 into the drug chamber 12. In one embodiment, an inlet 18a in the drug chamber 12 is coupled with a positive pressure device 20 for creating a positive pressure within the drug chamber 12. The drug chamber 12 also includes an outlet 18b for allowing the medicament to be delivered to a patient's lungs after the actuation mechanism 16 and the positive pressure device 20 are both activated. In another embodiment, the device 10 can include a connector (not shown) for coupling the outlet 18b in the drug chamber 12 to a tracheal tube. Where the inhalation device 10 includes a mouthpiece in communication with the outlet 18b, the connector can mate directly to the mouthpiece. The connector can be used alone with the inhalation device 10, or optionally it can be used in combination with the positive pressure device 20.

FIGS. 3-4 illustrate the inhalation device 100 of FIG. 1 as modified in accordance with the system shown in FIG. 2. Referring to FIG. 3 first, the air inlet 118a on the inhalation device 100 can be coupled to a positive pressure device 120 that is effective to create a positive pressure within the drug chamber 112 to force the medicament 114 through the outlet 118b in the drug chamber 112. A person skilled in the art will appreciate that virtually any positive pressure device can be used, including both manual and automatic devices for creating a positive pressure. By way of non-limiting example, FIG. 3 illustrates one exemplary embodiment of a positive pressure device 120 that is in the form of a standard ambulatory respiration device. The device 120 generally includes a balloon or bag member 121 having an outlet port 122 formed therein. Depending on the particular configuration of the inlet 118a formed in the inhalation device 100, the outlet 122 can either be directly connected to the inlet 118a, or it can be connected via a hollow elongate tube 124, as shown. The hollow elongate tube 124 should have a diameter that is sufficient to allow the terminals ends of the tube 124 to form a seal with the outlet 122 on the positive pressure device 120 and with the air inlet 118a on the inhalation device 100. Various connectors, tubing, or other mechanisms can also or alternatively be used to connect the positive pressure device 120 to the outlet 122 on the positive pressure device and/or to the inlet 118a in the inhalation device 100. For example, as is further shown in FIG. 3, an adapter 126 is provided and it extends between the outlet 122 on the positive pressure device 120 and the terminal end of the hollow elongate tube 124. Regardless of the type of connection formed between the positive pressure device 120 and the inhalation device 100, the connection should preferably be sealed to prevent leakage and to allow sufficient air pressure to be provided to the drug chamber 112 in the inhalation device 100.

The outlet 118b of the inhalation device 100 can be configured to deliver the medicament orally or to the patient's nasal passage, or it can be configured to connect to a tracheal tube. In the embodiment shown in FIG. 3, the inhalation device 100 includes a mouthpiece 113 that allows the patients to place their lips there around to form a seal. Thus, when the actuation mechanism 116 is depressed, and the positive pressure device 120 is activated, the medicament 114 is forced through the outlet 118b and the mouthpiece 113 to be delivered through the patient's mouth to their lungs. A person skilled in the art will appreciate that the mouthpiece 113 can have virtually any configuration, and that the mouthpiece 113 can alternatively be configured to deliver the medicament to a patient's nasal passage.

In use, a medicament 114 is placed within the drug chamber 112, and the actuation mechanism 116 is depressed to release the medicament from the capsule. The balloon or bag 121 is compacted or squeezed to blow air through the outlet 122 and the hollow elongate tube 124 and into the inlet 118a of the inhalation device 100, thereby creating a positive pressure within the drug chamber 112 to force the medicament 114 out through the outlet 118b and into the patient's lungs. In the embodiment shown in FIG. 3, the medicament 114 flows through the mouthpiece 113 and into the patient's mouth.

In an another embodiment, shown in FIG. 4, the inhalation device 100 can be adapted to connect to a tracheal tube. By way of non-limiting example, FIG. 4 illustrates a generally hollow connector 130 having first and second ends 130a, 130b that extend between the outlet 118b of the inhalation device 100 and a tracheal tube (not shown). The first end 130a can have a variety of configurations depending on the configuration of the outlet 118b in the inhalation device 100. In the illustrated embodiment, however, the first end 130a is adapted to mate to the mouthpiece 113, and more particularly it fits around the mouthpiece 113 to form a seal therewith. The second end 130b is similarly configured to mate to a tracheal tube, and it preferably simply slides over the tube to form a seal therewith.

In use, the connector 130 is mated to the mouthpiece 113 and a tracheal tube. The actuation mechanism 116 is then depressed, and the patient either inhales to cause the medicament 114 to flow through the mouthpiece 113 and the connector 130 and into a tracheal tube. In an alternative embodiment, where the patient cannot inhale, the connector 130 can be used in combination with the positive pressure device 120.

A person skilled in the art will appreciate that, while FIGS. 3 and 4 illustrate an inhalation device that is similar to the Handihaler™, a variety of inhalation devices can be used with the present invention. Moreover, the configuration of the inhalation device 10, and in particular the drug chamber 12 and the actuation mechanism 16 of the inhalation device 10, can vary depending on the configuration of the medicament 14. For example, in one embodiment the medicament 14 can be contained within a capsule or other receptacle that is placed within the drug chamber 12. The actuation mechanism 16 can thus be effective to puncture the capsule or otherwise cause the capsule to release the medicament 14 therefrom. In another embodiment, the medicament does not need to be encapsulated, but rather it can be contained outside of the drug chamber 12. The actuation mechanism can thus be configured to release a predetermined dosage of the medicament 14 into the drug chamber 12. A person skilled in the art will appreciate that the inhalation device 10 can have a variety of configurations, and that virtually any inhalation device can be used with the present invention. Certain modifications may be necessary, however, depending on the particular inhalation device used.

In an exemplary embodiment, the inhalation device 10 is preferably adapted to deliver a dry powder medicament. By way of non-limiting example, suitable dry powder inhalers for use with the methods and devices of the present invention include, but are not limited to, capsule devices such as Spinhaler™, Rotahaler™, Handihaler™, Aerohaler™, Eclipse™, Turbospin™ and the Flowcaps™ inhaler, and multidose inhalers such as Accuhaler™, Turbuhaler™, Ultrahaler™, Diskhaler™, Novoliser™, Easyhaler™, Taifun™, Clickhaler™, Twisthaler™ and Aspirair™.

One of ordinary skill in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

1. A device for delivering a dry powder medicament to the lungs, comprising:
an inhalation device having
a drug chamber having an inlet and an outlet, and
an actuation mechanism effective to release a medicament into the drug chamber;
a hollow elongate tube having a first end coupled to the inlet in the chamber and a second end; and
a positive pressure generating device coupled to the second end of the hollow elongate tube and effective to selectively pump the medicament from the chamber through the outlet in the inhalation device.

2. The device of claim 1, further comprising a mouthpiece coupled to the outlet in the drug chamber.

3. The device of claim 2, further comprising a mouthpiece connector having a first end adapted to couple to the mouthpiece and a second end adapted to couple to a tracheal tube in communication with a patient's lungs.

4. The device of claim 1, wherein the positive pressure generating device comprises an artificial resuscitator bag.

5. A device for delivering a dry powder medicament to the lungs, comprising:
an inhalation device having
a drug chamber having an inlet and an outlet, and
an actuation mechanism effective to release a medicament into the drug chamber; and
a mouthpiece connector having a first end adapted to couple to the outlet in the inhalation device and a second end adapted to couple to a tracheal tube in communication with a patient's lungs.

6. The device of claim 5, further comprising a mouthpiece coupled to the outlet in the drug chamber and the first end of the mouthpiece connector.

7. The device of claim 5, further comprising a positive pressure generating device coupled to the drug chamber inlet and effective to selectively pump the medicament from the chamber through the outlet in the inhalation device.

8. The device of claim 7, wherein the positive pressure generating device comprises an artificial resuscitator bag.

9. A method for delivering a dry powder medicament to the lungs, comprising:
activating an actuation mechanism on an inhalation device to release a medicament into a chamber in the inhalation device; and
activating a positive pressure generating device having a hollow elongate tube extending therefrom and in communication with an inlet in the chamber of the inhalation device to thereby cause the medicament within the chamber to flow through an outlet in the chamber and into a patient's lungs.

10. The method of claim 9, further comprising the step of coupling the outlet in the inhalation device to a tracheal tube in communication with a person's lungs such that, upon activation of the actuation mechanism and the positive pressure generating device, the medicament flows through the outlet and into the tracheal tube to be delivered to the patient's lungs.

11. The method of claim 9, wherein the inhalation device includes a mouthpiece that is in communication with the outlet in the drug chamber.

12. The method of claim 11, further comprising the step of coupling the mouthpiece to a tracheal tube in a patient.

13. The method of claim 12, wherein the mouthpiece is coupled to the tracheal tube by a mouthpiece connector.

14. The method of claim 9, wherein the positive pressure generating device is coupled to the inlet in the chamber of the inhalation device by a hollow elongate tube extending therebetween.

15. The method of claim 9, wherein the positive pressure generating device comprises an artificial resuscitator bag.

16. A kit for use with an inhalation device having a drug chamber adapted to receive a medicament, and an actuation mechanism effective to release a medicament into the chamber, the kit comprising at least one of:
a positive pressure generating device having a hollow elongate tube coupled thereto and including a terminal end that is adapted to mate to an inlet in communication with a drug chamber in an inhalation device;
a hollow elongate tube adapted to connect a positive pressure generating device to an inlet in communication with a drug chamber in an inhalation device; and
a mouthpiece connector having a first end that is adapted to couple to a mouthpiece formed on an inhalation device, and a second end that is adapted to couple to a tracheal tube in communication with a patient's lungs.
